(19) United States
(12) Patent Application Publication
(10) Pub. No.: US 2019/0167344 A1
(43) Pub. Date: Jun. 6, 2019

(54) MEDICAL EXAMINATION AND TREATMENT DEVICE FOR PRODUCING AND/OR VISUALISING AND/OR TREATING ABLATION LESIONS ON TISSUE OR VESSEL SURFACES

(71) Applicant: FELIX BOURIER, MUNICH (DE)

(72) Inventor: FELIX BOURIER, MUNICH (DE)

(21) Appl. No.: 16/157,454

(22) Filed: Oct. 11, 2018

(30) Foreign Application Priority Data
Oct. 24, 2017 (DE) 102017009865.4

(51) Int. Cl. A61B 18/14

(52) U.S. Cl. CPC A61B 18/1492; A61B 2018/00351

(57) ABSTRACT

The invention relates to a medical examination and treatment device for producing and/or visualising and/or treating ablation lesions on tissue or vessel surfaces, in particular in the heart, comprising at least one catheter tube, which forms an elongate inner cavity within which at least one HF ablation wire is guided, wherein the catheter tube and/or the HF ablation wire is controllable in an open and/or closed-loop manner by an open and/or closed-loop control device such that heat and/or cold can act on the tissue or vessel surfaces in a targeted manner along the tissue or vessel surfaces by means of the HF ablation wire, in particular in order to obliterate said surfaces at least in part, at least one measurement and/or recording system designed and provided to measure electrical impulses of the tissue or vessel surfaces such that electrical impulses can additionally be measured at various sites on the tissue or vessel surfaces, in particular during treatment, more particularly simultaneously, wherein at least one means for measuring, monitoring and/or modifying electrical impulses and/or for measuring, monitoring and/or modifying a time delay of an electrical impulse sequence at at least two sites on the tissue or vessel surfaces, in particular in order to prevent undesirable damage to the tissue or vessel surfaces.

The present invention relates to a medical examination and treatment device for producing and/or visualising and/or treating ablation lesions on tissue or vessel surfaces, in particular in the heart.

Taking the example of the heart, a catheter ablation refers to a method in which certain forms of cardiac arrhythmia are cured permanently. The origins of this treatment procedure date back to as early as the 80s. Since then, enormous steps have been made in this area.

Nowadays, numerous cardiac departments carry out the procedure.

To understand the principle of catheter ablation, some basic knowledge of the functioning of the heart is required: The heart consists of four chambers—two atria and two main chambers. The heartbeat is generated by electrical impulses originating from a special site in the right atrium. Starting from this “sinus node”, the electrical impulses propagate to the heart chambers via the atria and the atrioventricular node (AV node) and cause the heart muscle to contract (cardiac conduction system).

If there are additional defective conduction paths or sites in the heart muscle tissue that trigger further excitations, tachycardia attacks or persistent tachycardia can occur.

These can be treated by doctors by means of “catheter ablation”. In this process, doctors obliterate either the starting point of the additional heartbeats or the abnormal conduction paths, depending on the underlying cause.

In most forms of cardiac arrhythmia, the doctor first tries to treat the condition with medication. If this therapy is unsuccessful, patients suffering from certain types of cardiac arrhythmia can be permanently cured of their complaints using catheter ablation.

Generally, catheter ablation is carried out as part of an electrophysiology study (EP study) in hospital. The standard procedure is high-frequency ablation. The principle of this involves the catheter tip emitting heat into the tissue at a precise location. Other ablation methods use cold (cryoablation).

As with a heart catheter or an EP study, catheter ablation is carried out under local anaesthetic. The patient is conscious. As necessary, the doctor prescribes analgesics and sedatives. In the EP study, the doctor first studies the exact nature of the cardiac arrhythmia and the site where they originate. Next, the doctor uses the ablation catheter to intentionally create small scars measuring a few millimetres in the heart tissue in order to prevent the cardiac arrhythmia occurring or being transmitted. After obliteration, the doctor may test whether the cardiac arrhythmia can still be triggered by electrical impulses. The duration of the operation varies widely and is very difficult to predict. It may last between two and six hours, or even longer in certain cases. For the doctor in charge, however, catheter ablation is a procedure that places great requirements on the doctor themselves in terms of their ability to react.

Specifically, during catheter ablation a measurement and/or recording system preferably continuously measures the electrical impulses at various sites, in particular at sites at which the ablation is to be carried out and/or at sites having particularly high electrical conductivity. It is therefore possible, for example, to take measurements at the sinus node or in the region thereof and/or at the AV node or in the region thereof and/or in the region of the conduction system of the heart chambers. Specifically, the surgical risk is high since obliteration at one site, for example within the heart, may result in a sequence signal present in the heart as a result of the conduction paths, in particular the neural pathways, to be delayed or even to completely fail to materialise.

The reason behind this sequence signal and its generation is that, upon excitation, for example excitation of the sinus node, the AV node in the heart is excited similarly following a time delay. The excitation of the AV node then forms a sequence signal following the excitation signal of the sinus node. If this sequence signal at said AV node remains below an amplitude threshold or the sequence signal is delayed longer than a sequence time range set by the surgeon, the surgeon must abort the obliteration as quickly as possible to prevent damage to the neural pathways in the heart. In addition, the demands on the ability of the treatment personnel to react are even greater since their reaction time must consistently be at most within the seconds range throughout the entire operation.

In the past, operations of this kind have proven challenging, in particular depending on the surgeon. Specifically, if the surgeon is unable to constantly maintain this required reaction time in relation to aborting or changing the ablation procedure throughout this operation, which lasts several hours, serious damage to the heart muscle and its neural pathways can be expected.

The inventor has now developed an entirely new apparatus that can completely prevent human error, i.e. human error on the part of the surgeon.

Firstly, the medical examination and treatment device being proposed here for producing and/or visualising and/or treating ablation lesions on tissue or vessel surfaces, in particular in the heart, relates to at least one catheter tube, which forms an elongate inner cavity within which at least one HF ablation wire is guided, wherein the catheter tube and/or the HF ablation wire is controllable in an open and/or closed-loop manner by an open and/or closed-loop control device such that heat and/or cold can act on the tissue or vessel surface in a targeted manner along the tissue or vessel surfaces by means of the HF ablation wire, in particular in order to obliterate said surfaces at least in part.

In addition, the medical examination and treatment device being proposed here comprises at least one measurement and/or recording system designed and provided to measure electrical impulses of the tissue or vessel surface such that electrical impulses can be measured at various sites on the tissue or vessel surfaces, in particular during treatment, more particularly simultaneously.

According to the invention, the examination and treatment device comprises at least one means for measuring, monitoring and/or modifying electrical impulses and/or for measuring, monitoring and/or modifying a time delay of an electrical impulse sequence at at least two sites on the tissue or vessel surfaces, in particular in order to prevent undesirable damage to the tissue or vessel surfaces.

In this respect, a preferably fully automatic system is proposed, on the basis of which the means being proposed here replaces the reaction time of the surgeon. For example, the means being proposed here has a reaction time in the microsecond or millisecond range after a prohibited time delay of an electrical impulse sequence is detected. A range of at most 600 ms has proven advantageous, for example.

Therefore, an electrical impulse sequence is always defined as an impulse sequence of two in particular dependent impulses that is specified over time and is triggered by a first impulse, for example at the sinus node, and ends in a sequence signal, for example at the AV node.

According to at least one embodiment, the medical examination and treatment device for producing and/or visualising and/or treating ablation lesions on tissue or vessel surfaces, in particular in the heart, comprises at least one catheter tube, which forms an elongate inner cavity within which at least one HF ablation wire is guided, wherein the catheter tube and/or the HF ablation wire is controllable in an open and/or closed-loop manner by an open and/or closed-loop control device such that heat and/or cold can act on the tissue or vessel surfaces in a targeted manner along the tissue or vessel surfaces by means of the HF ablation wire, in particular in order to obliterate said surfaces at least in part.

In addition, the medical examination and treatment device comprises at least one measurement and/or recording system designed and provided to measure electrical impulses of the tissue or vessel surfaces such that electrical impulses can be measured at various sites on the tissue or vessel surfaces, in particular during treatment, more particularly simultaneously.

According to the invention, the medical examination and treatment device comprises at least one means for measuring, monitoring and/or modifying electrical impulses and/or for measuring, monitoring and/or modifying a time delay of an electrical impulse sequence at at least two sites on the tissue or vessel surfaces, in particular in order to prevent undesirable damage to the tissue or vessel surfaces. In this case, at least one, for example precisely one, electrical impulse is preferably assigned to each site on the tissue or vessel surfaces. As a result, electrical impulses are constantly measured at different sites within a predetermined time frame in order to test neural conductivity, for example of the heart.

According to at least one embodiment, the means is designed and provided to reduce or completely interrupt an (electrical) energy supply to the HF ablation wire if it is noted that a time delay in the electrical impulse sequence exceeds a predeterminable threshold and/or if it is noted that an expected sequence signal either has completely failed to materialise or is measured within the predetermined time frame but is below a limit amplitude.

According to at least one embodiment, the measurement and/or recording system is connected to the tissue or vessel surface such that at least one logical or haptic measurement input is installed in the measurement and/or recording system and/or means per measurement point on the tissue or vessel surfaces, the means being connected to each measurement input for data transmission.

In this respect, the means being described here is wired between the open and closed-loop control device and the measurement and/or recording system for data transmission.

In this case, a logical measurement input in the measurement and/or recording system is preferably implemented solely by a logical algorithm stored in the measurement and/or recording system and/or in the means. For example, the measurement and/or recording system and/or the means has just one actual measurement input, a circuit logic and/or a program logic being stored within the measurement and/or recording system and/or within the means and making it possible for the, for example, one sole signal from the logical measurement input to be read out and converted and/or deconstructed in such a way that the time delay of the electrical impulse sequence can be measured and/or calculated and/or deduced therefrom.

In the process, a haptic measurement input is one that actually constitutes a separate physical measurement input for each signal, for example, in the measurement and/or recording system and/or in the means. Each haptic measurement input can thus be uniquely assigned to a single measurement point on the heart.

According to at least one embodiment, the means comprises at least one monitoring box, this monitoring box being connected to both the open and/or closed-loop control device and the measurement and/or recording system for data transmission.

In this respect, said monitoring box is a haptic element. The monitoring box can comprise the above-described logical or at least two haptic measurement inputs.

According to at least one embodiment, the means compares selected measured impulses, which are each assigned, preferably uniquely, to different sites within or along the tissue or vessel surfaces, in terms of their respective trigger times, durations and/or amplitude levels.

According to at least one embodiment, the means displays at least one optical and/or acoustic alarm signal on its screen when the predetermined time delay is exceeded. According to at least one embodiment, the means indicates the precise site on the tissue or vessel surfaces at which an impulse time has changed and/or is absent.

According to at least one embodiment, various tissue or vessel surface types, for example a human heart and/or another tissue, are stored in the means, wherein, before and/or during treatment, a clinician can select the vessel types to be treated. If a vessel type of this kind is now selected, the means can load appropriate minimum and/or maximum impulse time ranges between two measured signals, and so the means is able, during the operation, to compare each measured impulse time sequence with the impulse time sequence stored in the means.

It is also conceivable for various surgeon IDs to be stored in the means. For example, the surgeon inputs their corresponding ID into the means at the start of the examination. After the ID has been entered, the means can load the maximum impulse sequence time delay accordingly desired by the surgeon. A maximum amplitude deviation can also be loaded.

In addition, it is conceivable that, after the surgeon has input their ID, they input a time delay and/or amplitude deviation that has not yet been stored. For example, the means then calculates a mean time delay and/or a mean amplitude deviation for a particular stored vessel and/or tissue type. This average can be used by the surgeon to calculate an average deviation. When the average deviation is exceeded, therefore, conclusions can be drawn on the expected average damage to the vessel and/or tissue type.

If the stored impulse time sequence deviates from the measured impulse time sequence by more than 5%, preferably by more than 8%, the means can output a warning signal that can be either seen or heard. However, it is also conceivable for the means to additionally or alternatively interrupt any power supply to the HF ablation wire, such that any treatment has to be aborted. This ensures patient safety and thus helps limit the damage already caused by the ablation.

According to at least one embodiment, at least two impulse measuring sites along the tissue or vessel surfaces can be input into the means and/or the means detects said impulse measurement sites autonomously before and/or during treatment, in particular by means of suitable measurement probes.

As a result, it is possible for the means to not only indicate whether a time lag between the two individual impulses, for example between the sinus node impulse and the AV node impulse or between the AV node impulse and the heart chamber impulse, has been exceeded, but also for the means to additionally indicate the particular measurement site of each impulse along the tissue or vessel.

According to at least one embodiment, if the time delay between two impulses at two different measurement sites on the tissue or vessel surfaces is exceeded, the means issues at least one treatment recommendation for the clinician.

The treatment recommendation may be a notification on the screen of the means stating that any treatment must be stopped immediately, or indicating a remaining time for the treatment until which the treatment can still be carried out within reasonable limits.

The above-described invention will be described in more detail below on the basis of an embodiment and the associated drawings.

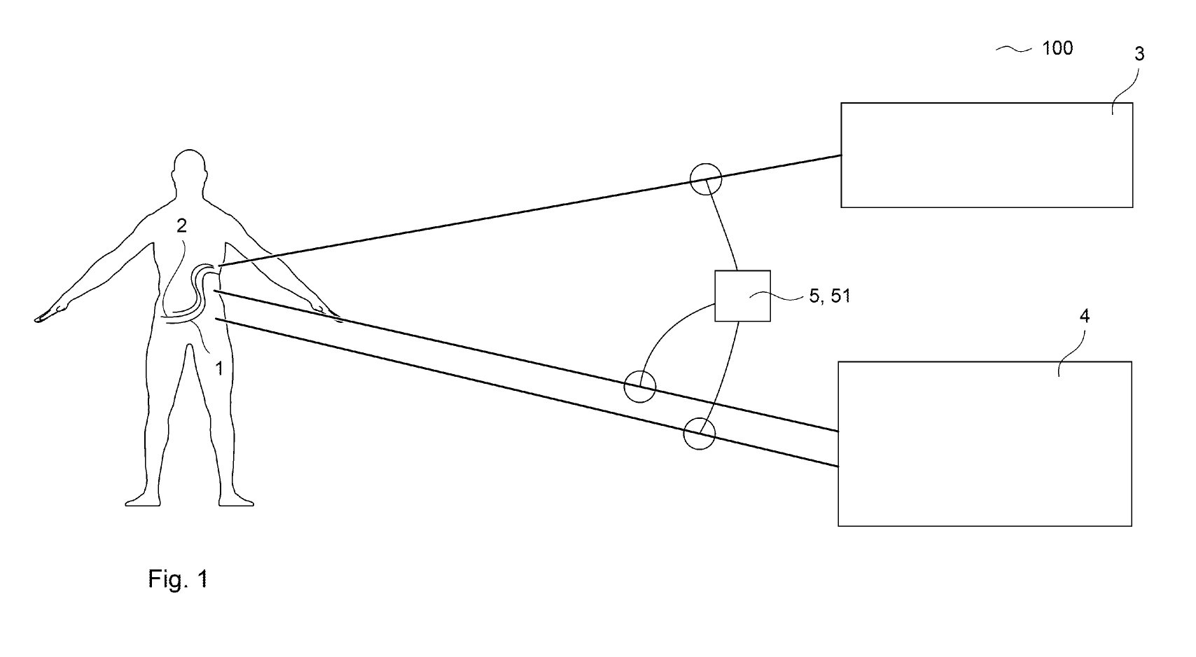

FIG. 1 shows a medical examination and treatment device 100 for producing and/or visualising and/or treating ablation lesions on tissue or vessel surfaces, in particular in the heart.

The medical examination and treatment device 100 being described here comprises a catheter tube 1, which forms an elongate inner cavity within which at least one HF ablation wire 2 is guided, wherein the catheter tube 1 and/or the HF ablation wire 2 is controllable in an open and/or closed-loop manner by an open and/or closed-loop control device 3 such that heat and/or cold can act on the tissue or vessel surfaces in a targeted manner along the tissue or vessel surfaces by means of the HF ablation wire 2, in particular in order to obliterate said surfaces at least in part.

In addition, the medical examination and treatment device 100 comprises at least one measurement and/or recording system 4 designed and provided to measure electrical impulses of the tissue or vessel surfaces such that electrical impulses can be measured at various sites on the tissue or vessel surfaces, in particular during treatment, more particularly simultaneously.

Furthermore, the examination and treatment device 100 comprises at least one means 5 for measuring, monitoring and/or modifying electrical impulses and/or for measuring, monitoring and/or modifying a time delay of an electrical impulse sequence at at least two sites on the tissue or vessel surfaces, in particular in order to prevent undesirable damage to the tissue or vessel surfaces.

In this case, the means 5 shown in FIG. 1 is designed and provided to reduce or completely interrupt an energy supply to the HF ablation wire 2 if it is noted that a time delay in the electrical impulse sequence exceeds a predeterminable threshold and/or if it is noted that an expected sequence signal has completely failed to materialise. It can also be seen in FIG. 1 that the means 5 is in the form of a separately arranged monitoring box 51. This monitoring box comprises at least two signal inputs, each signal input being assigned to one site on the tissue or vessel surface. Consequently, the monitoring box 51 measures at least two signals, the two measured signals thus representing an impulse sequence, the second signal being measured after a time delay compared with the first signal.

It can also be seen that the monitoring box 51 is connected to the HF generator for signal transmission and an energy supply of the HF generator to the ablation wire 2 can be reduced, increased or completely interrupted depending on the settings set by the clinician.

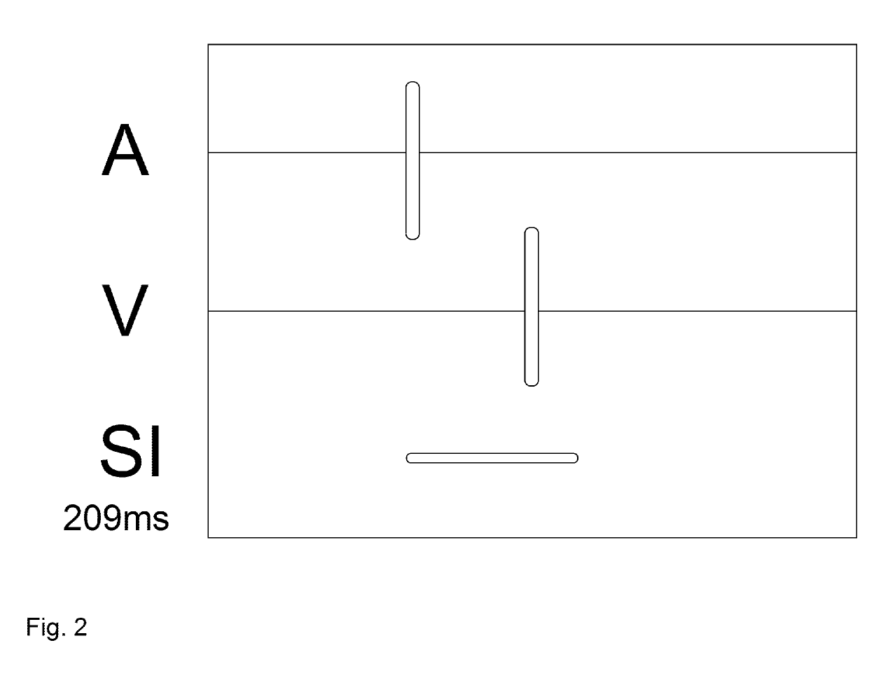

FIG. 2 shows that two measurement points “A” (corresponding for example to the sinus node) and “V” (corresponding for example to the AV node) have been triggered on the display of the means 5 at different times in the horizontal direction. The horizontal bar below these two signals is shown by the abbreviation “SI”. This delay bar “SI” can represent a maximum time delay between the two signals, predetermined by the user. If, in the vertical direction, the two signals “A” and “V” are still within the range in the horizontal direction defined by the signal “SI”, the tissue, in particular the conductive tissue, between the measurement points “A” and “V” has not yet been damaged or has at least not yet been significantly damaged. Therefore, as long as these two signals remain within the horizontal time bar, the operation or study may continue as normal.

However, if a horizontal distance between the two signals “A” and “V” is greater than a time length of the specified distance “SI”, as shown in FIG. 2, i.e. is greater than 209 ms for example, the means 5 can be set such as to interrupt or at least reduce the power supply to the ablation wire 2. As described above, a visual or an acoustic alarm function is also conceivable.

The invention is not limited by the description and the embodiments; instead, the invention covers any novel feature and any combination of features (including in particular any combination of features in the claims), even if this feature or combination is not explicitly disclosed in the claims or in the embodiment.

### LIST OF REFERENCE NUMERALS

1 Catheter tube
2 HF ablation wire
3 Open and/or closed-loop control device
4 Measurement and/or recording system
5 Means
51 Monitoring box
100 Medical examination and treatment device

1. Medical examination and treatment device (100) for producing and/or visualising to and/or treating ablation lesions on tissue or vessel surfaces, in particular in the heart, comprising
at least one catheter tube (1), which forms an elongate inner cavity within which at least one HF ablation wire (2) is guided, the catheter tube (1) and/or the HF ablation wire (2) being controllable
in an open and/or closed-loop manner by an open and/or closed-loop control device (3) such that heat and/or cold can act on the tissue or vessel surfaces in a targeted manner along the tissue or vessel surfaces by means of the HF ablation wire (2), in particular in order to obliterate said surfaces at least in part,
at least one measurement and/or recording system (4) designed and provided to measure electrical impulses of the tissue or vessel surfaces such that electrical impulses can additionally be measured at various sites on the tissue or vessel surfaces, in particular during treatment, more particularly simultaneously,
characterised by
at least one means (5) for measuring, monitoring and/or modifying electrical impulses and/or for measuring, monitoring and/or modifying a time delay of an electrical impulse sequence at at least two sites on the tissue or vessel surfaces, in particular in order to prevent undesirable damage to the tissue or vessel surfaces.

2. Examination and treatment device (100) according to claim 1,
characterised in that
the means (5) is designed and provided to reduce or completely interrupt an energy supply to the HF ablation wire (2) if it is noted that a time delay in the electrical impulse sequence exceeds a predeterminable threshold and/or if it is noted that an expected sequence signal has completely failed to materialise.

3. Examination and treatment device (100) according to either claim 1 or claim 2,
characterised in that
the measurement and/or recording system (4) is connected to the tissue or vessel surfaces such that one logical or haptic measurement input is installed in the measurement and/or recording system (4) per measurement point on the tissue or vessel surfaces, the means (5) being connected to each measurement input for data transmission.

4. Examination and treatment device (100) according to at least one of the preceding claims,
characterised in that
the means (5) comprises at least one monitoring box (51), this monitoring box (51) being connected to both the open and/or closed-loop control device (3) and the measurement and/or recording system (4) for data transmission.

5. Examination and treatment device (100) according to at least one of the preceding claims,
characterised in that
the means (5) compares selected measured impulses, which are each assigned, preferably uniquely, to different sites on the tissue or vessel surfaces, with one another in terms of their respective trigger times, durations and/or amplitude levels.

6. Examination and treatment device (100) according to at least one of the preceding claims,
characterised in that
the means (5) displays at least one optical and/or acoustic alarm signal on its screen when the predetermined time delay is exceeded.

7. Examination and treatment device (100) according to at least one of the preceding claims,
characterised in that
the means (5) indicates the precise site on the tissue or vessel surfaces at which an impulse time has changed and/or is absent.

8. Examination and treatment device (100) according to at least one of the preceding claims,
characterised in that
various tissue or vessel surface types, for example a human heart and/or another tissue, are stored in the means (5) and, before and/or during treatment, a clinician can select the vessel types to be treated.

9. Examination and treatment device (100) according to at least one of the preceding claims,
to characterised in that
at least two impulse measurement sites along the tissue or vessel surfaces can be input into the means (5) and/or the means (5) can detect said impulse measurement sites autonomously before and/or during treatment, in particular by means of suitable measurement probes.

10. Examination and treatment device (100) according to at least one of the preceding claims,
characterised in that
if the time delay between two impulses at two different measurement sites on the tissue or vessel surfaces is exceeded, the means (5) issues at least one treatment recommendation for the clinician.
